(19) United States
(12) Patent Application Publication
(10) Pub. No.: US 2006/0263760 A1
(43) Pub. Date: Nov. 23, 2006

(54) Screening method of substance with antidepressant action

(75) Inventors: Suguru Kawato, Tokyo (JP); Yuki Oishi, Tokyo (JP)

Correspondence Address: OLIFF & BERRIDGE, PLC, P.O. BOX 19928, ALEXANDRIA, VA, 22320, US

(73) Assignee: THE UNIVERSITY OF TOKYO, Tokyo (JP)

(21) Appl. No.: 11/281,509

(22) Filed: Nov. 18, 2005

(30) Foreign Application Priority Data
May 23, 2005 (JP) 2005-149864

(51) Int. Cl. C12Q 1/00

(52) U.S. Cl. 435/4

(57) ABSTRACT

There is provided a rapid and simple screening method of a substance with an antidepressant action. The screening method of a substance with an antidepressant action in accordance with the invention includes a cell preparation step for preparing a cell showing a predetermined electrophysiological response on a given stimulation and modifying the electrophysiological response when in contact to a depression-related substance, a candidate substance treatment step for putting a candidate substance as a screening subject and the depression-related substance in contact to the prepared cell and assaying the electrophysiological response of the cell, a control treatment step for putting the depression-related substance in contact to the prepared cell and assaying the modification of the electrophysiological response, and an assessment step for assessing whether or not the modification of the electrophysiological response as assayed at the control treatment step is suppressed in the assay results obtained at the candidate substance treatment step.

### BACKGROUND OF THE INVENTION

The present invention relates to a screening method of a substance with an antidepressant action. Particularly, the invention relates to the assessment of cellular electrophysiological response.

The number of patients diagnosed as depression is likely to increase in modern societies under higher stresses. Therefore, the development of so-called antidepressant capable of ameliorating the symptoms of depression has been desired.

As such antidepressant, for example, antidepressants for elevating serotonin concentration and for activating serotonin receptors have mainly been developed, on the basis of the finding that monoamine-series neurotransmitters such as serotonin are deficient in the brains of patients with depression.

Additionally in the related art, for example, a screening method of an active substance applicable as such antidepressant has been used, which includes giving stress to an experimental animal such as mouse by so-called forced swimming method and tail suspension method to induce depression symptoms, then administering a candidate active substance applicable as antidepressant to the animal, and examining the antidepressant effect (the effect on the amelioration of depression symptoms) (Karolewicz B., Paul IA. European Journal of Pharmacology 415, 197-201 (2001)).

Reports have been issued in recent years, telling the presence of substances associated with depression, such as corticoid as one of adrenocorticoid hormones other than monoamine-series neurotransmitters. Therefore, subjects for screening for substances with antidepressant actions are increasingly diverse.

In such current status, the screening method using experimental animals in the related art as described above requires much time and cost, inefficiently.

### SUMMARY OF THE INVENTION

The invention has been made, taking account of the problems described above. It is an object of the invention to provide a method for rapidly screening for a substance with an antidepressant action in a simple manner.

So as to solve the problems in the related art, the screening method of a substance with an antidepressant action as one embodiment of the invention includes a cell preparation step for preparing a cell showing a predetermined electrophysiological response on a given stimulation and modifying the electrophysiological response when in contact to a depression-related substance, a candidate substance treatment step for putting a candidate substance as a screening subject and the depression-related substance in contact to the prepared cell and assaying the electrophysiological response of the cell, a control treatment step for putting the depression-related substance in contact to the prepared cell and assaying the modification of the electrophysiological response, and an assessment step for assessing whether or not the modification of the electrophysiological response as assayed at the control treatment step is suppressed in the assay results obtained at the candidate substance treatment step.

In accordance with the invention, a substance with an antidepressant action can be screened in a rapid and simple manner by assessing whether or not the modification of the electrophysiological response induced in the cell by a depression-related substance is suppressed.

### BRIEF DESCRIPTION OF THE DRAWINGS

FIG. 1 is an explanatory view showing one waveform example of the accumulated potential assayed by the screening method in one embodiment of the invention.

FIG. 2 shows graphs depicting one example of an effect of a depression-related substance reducing the long-term potentiation level, as measured using the screening method in one embodiment of the invention.

FIG. 3 shows graphs depicting one example of an effect of a substance with an antidepressant action recovering the long-term potentiation level, as measured using the screening method in one embodiment of the invention.

### DESCRIPTION OF THE PREFERRED EMBODIMENTS

The screening method of an antidepressant action in one embodiment of the invention (referred to as the screening method hereinbelow) is now described below. Herein, the screening method of a substance with an antidepressant action is not limited to the following embodiment.

First, the screening method is generally described as below. The screening method is made on the basis of a finding independently found by the present inventors.

The inventors made investigations and consequently found that when so-called glucocorticoid (corticosteron, cortisol, etc.) was put in contact to a cell with a corticoid receptor (glucocorticoid receptor, mineral corticoid receptor, etc.), an acute action to modify the electrophysiological response was induced in the cell in a very short time after the contact, compared with no contact. It is considered that the acute action simply reproduces the phenomenon of inducing a depression state via the increase of glucocorticoid in living bodies during stress response, at cellular level.

The inventors additionally found that when so-called female hormone (estrogen) is put in contact to a cell with a receptor of the hormone which modifies its electrophysiological response via glucocorticoid, the modification of the electrophysiological response in the cell via glucocorticoid is dissolved in a very short time after the contact. It is considered that this simply reproduces the antidepressant action of the female hormone, as suggested in the related art.

Based on the novel findings made by the inventors as described above that in a cell which shows an electrophysiological response on a given stimulation, the electrophysiological response is modified when the cell is in contact to a depression-related substance and the modification of the electrophysiological response due to the depression-related substance is suppressed when the cell is in contact to a substance with an antidepressant action, it is assessed in the screening method whether or not a candidate substance as a screening subject is a substance with an antidepressant action by assessing whether or not the candidate substance suppresses the modification of the electrophysiological response due to the depression-related substance.

Any depression-related substance can be used as long as the depression-related substance is a biological substance associated with depression symptoms or a derivative thereof with an action to modify the electrophysiological response of a cell. For example, there may be used so-called stress hormones known for its secretion being accelerated, or its concentration in blood or tissue being increased or decreased in a living body when the living body is exposed to stresses.

Specifically, corticoids for example glucocorticoid and mineral corticoid as adrenocorticoid hormones may be used as the depression-related substance. Corticosteron and cortisol are particularly preferably used.

Additionally, the substance with an antidepressant action means a substance suppressing the modification of an electrophysiological response induced in a cell when in contact with a depression-related substance, namely a substance with an action dissolving such modification. In this embodiment, the substance with an antidepressant action includes female hormones including estrogen such as estradiol, estron and estriol, and hexestrol and stilbestrol with the same physiological actions as those of female hormone.

The screening method is specifically described. The screening method includes a cell preparation step for preparing a cell showing a predetermined electrophysiological response on a given stimulation and modifying the electrophysiological response when in contact to a depression-related substance, a candidate substance treatment step for putting a candidate substance as a screening subject and the depression-related substance in contact to the prepared cell and assaying the electrophysiological response of the cell, a control treatment step for putting the depression-related substance in contact to the prepared cell and assaying the modification of the electrophysiological response, and an assessment step for assessing whether or not the modification of the electrophysiological response as assayed at the control treatment step is suppressed in the assay results obtained at the candidate substance treatment step.

At the cell preparation step, a cell is prepared for use as a subject for the assessment of the physiological response thereof when in contact to a depression-related substance or a candidate substance. For example, tissues taken from humans or animals, isolated primary cells, cells of established strains, or cells prepared by artificially treating these cells with genetic recombination procedures are prepared.

As such cell, any cell may be used with no specific limitation as long as the cell involves the modification of the electrophysiological response thereof in contact to a depression-related substance, compared with no such contact and also involves the suppression of the modification of the electrophysiological response when in contact to a substance with an antidepressant action. For example,-cells with receptors of depression-related substances and cells with receptors of substances with antidepressant actions may be used.

More specifically, nerve cells contained in brain tissue and other nerve tissues for example can preferably used as such cell. In case that brain tissue is to be used, for example, brain tissues from small animals such as rat and mouse may be used. In particular, extrapyramidal cells contained in cornu ammonis (CA) and granule cells contained in hippocampus dentate gyrus in a longitudinal cross-sectional section prepared by vertically cutting brain hippocampus region along the longitudinal direction, using a section preparation apparatus such as vibratome may preferably be used.

As the brain tissue section, further, a brain tissue section of a thickness within a range of about 150 to 400 μm may preferably be used. In particular, a brain tissue section of a thickness of about 400 μm may preferably be used. The reason is as follows. When the thickness of a brain tissue section is smaller than about 150 μm, most of nerve cells contained in the brain tissue section are damaged severely at the cell bodies or dendrites during the preparation of the section. Thus, the resulting section is hardly used as normal tissue. When the thickness of a brain tissue section is larger than about 400 μm, oxygen supply from a solution charging the brain tissue section therein to the nerve cells inside the brain tissue section gets poor, so that the resulting brain tissue section is hardly used as normal tissue. In case that isolated nerve cell is used, for example, nerve cell cultured in a culture medium of a given composition in a given culture substratum such as commercially available plastic dish for a given period of time should be prepared.

At the candidate substance treatment step, the candidate substance as a screening subject and a depression-related substance are put in contact to the cell prepared at the cell preparation step, to assay the electrophysiological response of the cell in contact to the candidate substance and the depression-related substance to a given stimulation.

Any candidate substance can be used with no specific limitation, as long as the candidate substance can be used as a screening subject for the assessment as to whether or not the candidate substance exerts an antidepressant action. For example, appropriate compounds such as substances reportedly having any antidepressant action at animal experiments and derivatives thereof can be used, irrespective of whether or not they have known chemical structures or properties (for example, actions toward living bodies or cells) or whether or not they are natural substances or artificially synthesized substances.

At the candidate substance treatment step, first, the cell is put in simultaneous contact to the candidate substance and a depression-related substance. Specifically, a tissue section or an isolated cell is immersed in a solution containing both the candidate substance and a depression-related substance (referred to as stimulating solution) for a given period of time or the stimulating solution is sprayed on the vicinity of a cell contained in a tissue section or an isolated cell, using a glass pipette and the like, to put the cell contained in the tissue section or the isolated cell in contact to the candidate substance and the depression-related substance. The time period required for the contact of the cell to the candidate substance and the depression-related substance can appropriately be selected, depending on the object. For the assessment of a relatively slow response through gene expression, the time period is preferably several hours (for example, 3 hours) or more. For the assessment of a relatively acute response without gene expression, the time period is preferably several tens of minutes (for example, about 30 minutes).

When the stimulating solution is used, the concentration of a candidate substance in the stimulating solution is appropriately selected, with no specification. In case that the candidate substance is a substance existing in a living body, for example, a concentration in the living body (so-called physiological concentration) may preferably be used. Additionally, an appropriate concentration may be selected for the depression-related substance, depending on the type thereof. In case that corticosteron is used, for example, a relatively high concentration is preferable. 1 μM or more is preferably used. At a relatively low concentration, corticosteron likely exerts a stronger action as a nerve nutritious factor, compared with the action as a depression-related substance. The concentrations of these candidate substances and depression-related substances may be determined on the basis of the affinities thereof to a binding receptor. For example, a concentration close to the dissociation constant between such receptor and the candidate substances, or such receptor and the depression-related substances (so-called Kd value) may preferably be used.

The stimulating solution and the following other solutions may satisfactorily be circulated (perfused) in a given container containing the tissue sections and the isolated cells, using given pump apparatuses and the like or may be held in the given container in a batch-wise manner.

Any solvent dissolving the candidate substance and the depression-related substance may be used with no specific limitation, as long as the solvent can keep such cells viable. Aqueous solutions are preferably used, where for example the composition and concentrations of salts are adjusted to give an appropriate permeation pressure or the composition and concentrations of nutritious components (glucose, amino acids and the like) required for cellular metabolism are adjusted. At the candidate substance treatment step, then, a predetermined stimulation is given to the cell in contact to the candidate substance and the depression-related substance, to assay the electrophysiological response of the cell to the stimulation.

Any cell stimulating method may be used with no specific limitation, as long as the stimulating method triggers cellular electrophysiological response. For example, methods usable repeatedly such as electric stimulation and chemical stimulation to put the cell in contact to given substances are preferably used.

In case that nerve cell is used, specifically, an electric stimulation by inserting a glass electrode in the cell and a local stimulation by spraying a nerve transmitter to the vicinity of the cell using a glass pipette may be used. In case that a brain tissue section is used, particularly, an electric stimulation of a presynaptic fiber projecting in a never cell contained in the brain tissue section may preferably be used because much skill is not required therefor and because the electric stimulation can be carried out in a stable manner.

As the electrophysiological response, any electric properties of viable cell to given stimulation, including potential change of cellular membrane may be assayed. In case that a brain tissue section is used, for example, postsynaptic potential to an electric stimulation on presynaptic fiber and the change thereof can be assayed.

In case that a brain tissue section is used, specifically, so-called long-term potentiation or long-term depression representing the characteristic change of excitatory postsynaptic potential in association with the nerve cell contained in the brain tissue section may be assayed as one electrophysiological response to an electric stimulation at a given frequency.

In case that the long-term potentiation is assayed as the electrophysiological response, any method for inducing long-term potentiation may appropriately be selected with no specific limitation. For example, various high-frequency electric stimulation patterns to presynaptic fiber projecting in a nerve cell may be used. From the standpoint of relative simplicity and good reproducibility and the standpoint of the high frequency of the actual use in previous reports, a stimulation method of a high frequency, namely 100 stimulations per one second may preferably be used.

As the method for assaying electrophysiological response, additionally, methods for assaying cellular potential in one cell such as a glass electrode insertion method including inserting a glass electrode into a cell and a patch cramp method using a glass electrode, may be used.

In case that a brain tissue section is used, for example, a method for assaying the accumulated potential of nerve cells contained in the brain tissue section may specifically be used. Particularly, a method for assaying the accumulated potential using a glass electrode can be used preferably, by which presynaptic fiber can be stimulated highly efficiently and postsynaptic potential can be assayed at high sensitivity. When an assay method of the accumulated potential using a single electrode assay apparatus is selected as the assay method using a glass electrode, the single electrode assay apparatus may be a single electrode assay apparatus appropriately prepared or a commercially available such apparatus.

The distance between the stimulation electrode and a recording electrode is not specifically limited. Depending on the purpose, the distance can appropriately be selected. In case that a rat brain hippocampus is used, for example, the range of about 300 to 400 μm can be used as a preferable distance between the electrodes. Particularly, the distance is preferably 300 μm. This is because the stimulation electrode and a recording electrode are necessarily arranged in the hippocampus CA1 region and because in case that the distance between the electrodes is smaller than the range, for example, the assay results may sometimes be influenced by the stimulation electrode per se.

At the control treatment step, the same kind of depression-related substance as the depression-related substance in contact to the cell at the candidate substance treatment step is put in contact to the cell prepared at the cell preparation step, to assay the electrophysiological response to a given stimulation in the cell in contact to the depression-related substance.

At the control treatment step, specifically, the modification of the electrophysiological response due to the depression-related substance is verified in the cell prepared at the cell preparation step, which shows a predetermined electrophysiological response on a given stimulation with no contact to a depression-related substance and involves the modification of the electrophysiological response in contact to the depression-related substance.

At the control treatment step, specifically, a tissue section or an isolated cell is immersed in a solution (referred to a control solution hereinafter) with a composition same as that of the stimulating solution except that the control solution lacks the candidate substance (with no content of any candidate substance and with a content of the depression-related substance at the same concentration as in the stimulation solution) for a given period of time or the control solution is sprayed on the vicinity of the cell contained in the tissue section or the isolated cell, using a glass pipette or the like, to put the cell contained in the tissue section or in the isolated cell, in contact to the depression-related substance.

At the control treatment step, then, a predetermined stimulation is given to the cell in contact to the depression-related substance as in the candidate substance treatment step, to assay the electrophysiological response of the cell to the stimulation.

At the control treatment step, additionally, the electrophysiological response of a cell with no contact of any candidate substance or any depression-related substance may satisfactorily be assayed. At the control treatment step, specifically, the cell is immersed in a solution with a composition same as that of the stimulation solution except that the solution lacks both of the candidate substance and the depression-related substance for a given period of time or the solution is sprayed on the vicinity of the cell contained in the tissue section or the isolated cell, to assay the electrophysiological response of the cell contained in the tissue section or the isolated cell.

At the assessment step, the assay results of the electrophysiological response at the candidate substance treatment step and the assay results of the electrophysiological response at the control treatment step are compared to each other, to assess whether or not the modification of the electrophysiological response of the cell in contact to the depression-related substance is suppressed in the cell in contact to both the candidate substance and the depression-related substance.

At the assessment step, specifically, it is assessed whether or not the difference between the electrophysiological response level in the cell in contact to the depression-related substance and the electrophysiological response level in the cell without such contact is reduced or dissolved in the cell in contact to both the candidate substance and the depression-related substance, namely whether or not the cell shows an electrophysiological response closer to the response with neither contact to the candidate substance nor contact to the depression-related substance.

In case that compared with the response without any contact to either the candidate substance or the depression-related substance, the potential of the cellular membrane is modified by a given amount when in contact to the depression-related substance, it is assessed whether or not the given amount is reduced or dissolved when in contact to both the candidate substance and the depression-related substance, namely whether or not the potential of the cellular membrane with no contact to either any candidate substance or any depression-related substance is recovered.

In case that the level of the long-term potentiation is reduced when the cell is in contact to a depression-related substance by a given amount compared with no contact to either any candidate substance or a depression-related substance when the long-term potentiation is assayed using a brain tissue section, it is assessed whether or not the reduction of the long-term potentiation level is suppressed or dissolved when in contact to both the candidate substance and the depression-related substance, namely whether or not the level of the long-term potentiation is recovered.

In the screening method, a candidate group treatment group treated by the contact to both a candidate substance and a depression-related substance, a control treatment group treated by the contact to a depression-related substance without any contact to any candidate substance, and a non-treatment group with no contact to either a candidate substance or a depression-related substance may satisfactorily be prepared.

When plural brain tissue sections are prepared from a brain tissue collected from a single mouse at the cell preparation step in this case, for example, a part of the brain tissue sections is treated by a candidate substance, while another part of the brain tissue sections is treated by a control and an additional part of the brain tissue sections is treated by neither the candidate substance nor the control. At the assessment step, the electrophysiological responses of the brain tissue sections of these three groups are assayed. The results are compared together and assessed.

In case that an isolated cell is used, for example, cells with the same properties based on an animal or a tissue as cultured under the same conditions including passage number and culturing period are prepared at the cell preparation step. Among the cultured cells, a part of the cultured cells is treated by the candidate substance, while another part of the cultured cells is treated by the control and an additional part of the cultured cells is treated by neither the candidate substance nor the control. At the assessment step, the electrophysiological responses of the cultured cells of these three groups are assayed. The results are compared together and assessed.

A specific example of the assessment of substances with antidepressant actions by the screening method is described below. At the cell preparation step in the Example, the brain hippocampus of an adult rat was cut vertically along the longitudinal axis using vibratome (DSK ZERO1, manufactured by DOUSAKA EM K.K), to prepare plural longitudinal cross- sectional sections of a thickness of 400 μm.

In this Example, a part of the hippocampus sections is prepared as a candidate substance treatment group, while another part of the hippocampus sections is prepared as a control treatment group and an additional part of the hippocampus sections is prepared as non-treatment group.

These hippocampus sections were immersed in an artificial cerebrospinal fluid sufficiently saturated with a mixture gas of oxygen and carbon dioxide (95% oxygen gas, 5% carbon dioxide gas) and left to stand at room temperature for 1.5 hours or more under the purge of the mixture gas from the above over the hippocampus sections. Thereafter, the electrophysiological response was assayed as described below.

As the artificial cerebrospinal fluid, an aqueous solution containing 124 mM NaCl, 1.25 mM NaH₂PO₄.2H₂O, 5 mM KCl, 2 mM MgSO₄.7H₂O, 2 mM CaCl₂, 22 mM NaHCO₃, and 10 mM glucose was used.

At the cell preparation step, the prepared hippocampus sections were left to stand in a given assay chamber, where an artificial cerebrospinal fluid sufficiently saturated with the mixture gas was filled around the hippocampus sections. Then, a given single electrode assay apparatus was mounted on the hippocampus sections. As the single electrode assay apparatus, an apparatus equipped with a glass electrode (Sutter Instruments Corporation) with a combination of a capillary and a silver/silver chloride electrode and with a given amplifier (Amp) (DAM80, World Precision Instrument) was used.

Specifically, a given dipole electrode was arranged as a stimulation electrode on the presynaptic fibers in the hippocampus CA1 regions of the individual hippocampus sections. As an assay electrode, the glass electrode was arranged on the dendrites in the postsynaptic cells.

At the following steps, the artificial cerebrospinal fluid sufficiently saturated with the mixture gas was perfused in the inside of the chamber with the hippocampus sections therein at a flow rate of about 2 ml/minute, for all the treatments and assays at the steps.

At the cell preparation step, additionally, the presynaptic fibers in the CA1 regions of the hippocampus sections were repeatedly stimulated at an interval of 20 seconds, using the stimulation electrode, while excitatory postsynaptic potential induced by the electric stimulation was recorded as accumulated potential over about 30 minutes.

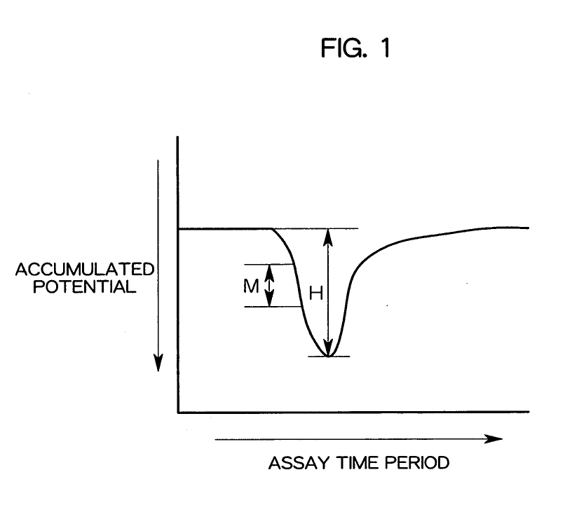

FIG. 1 shows one waveform example representing the accumulated potential recorded with one stimulation among electric stimulations carried out at the 20-second interval. In FIG. 1, the horizontal axis expresses assay time period, while the longitudinal axis expresses accumulated potential.

As shown in FIG. 1, the recorded accumulated potential is assayed as the absolute value H of the height of the waveform (namely, the difference between the base value and the peak value). Because the accumulated potential is the accumulated action potential, however, the record sometimes cannot express an exact value after high-frequency stimulation as described below. Therefore, it was determined to assess the dimension of the slope in the part M with the most rapid change of the accumulated potential on the stimulation [namely, the largest slope on the earlier side of the assay time period of accumulated potential waveform (on the left side)] (referred to as largest slope value of accumulated potential hereinbelow) on the waveform on FIG. 1.

At the cell preparation step, the stabilization of the assayed largest slope value of accumulated potential was first certified. Subsequently, the largest slope value of accumulated potential was assayed while gradually increasing the level of the loaded electric stimulation until the assayed largest slope value of accumulated potential was saturated (in other words, until the value reached a constant value). In case that the saturated largest slope value of accumulated potential just then was defined as the largest value (100%), the intensity of an electric stimulation to be loaded on the hippocampus sections from the stimulation electrode was adjusted to 50% of the largest value.

At the candidate substance treatment step, the accumulation potential of the hippocampus sections of the candidate substance treatment group was recorded at the electric stimulation intensity adjusted at the cell preparation step, by the repetitive stimulation method at the constant interval for 20 minutes. Then, the perfusion fluid was replaced with an artificial cerebrospinal fluid (namely, the stimulation solution) containing corticosteron (depression-related substance) and estradiol (a substance with an antidepressant action) dissolved in dimethylsulfoxide (DMSO) at a final 1 μM concentration and a final 1 nM concentration, respectively for continuous recording of the accumulated potential for another 30 minutes.

While recording the accumulated potential, further, the hippocampus sections were given a high-frequency stimulation at 100 stimulations/one second (namely, 100 Hz), to induce long-term potentiation and to subsequently record the accumulated potential over another 60 minutes.

At the control treatment step, the accumulated potential of the hippocampus sections of the control treatment group was recorded by the stimulation method at the 20-second interval over 20 minutes using the electric stimulation intensity adjusted at the cell preparation step, as in the candidate substance treatment step. Then, the perfusion fluid was replaced with an artificial cerebrospinal fluid (namely, the control solution) containing corticosteron dissolved in DMSO at a final concentration of 1 μM with no estradiol content, for further recording the accumulated potential for 30 minutes. The long-term potentiation induced by the 100-Hz high-frequency stimulation for one second was continuously recorded as the accumulated potential over 60 minutes.

At the control treatment step, the accumulated potential of the hippocampus sections of the non-treatment group was recorded by the stimulation method at the 20-second interval over 20 minutes, using the electric stimulation intensity adjusted at the cell preparation step. Then, the perfusion fluid was replaced with an artificial cerebrospinal fluid containing neither corticosteron nor estradiol but containing the same amount of DMSO as in the stimulation solution and in the control solution, for recording the accumulated potential for 30 minutes. Then, a high-frequency stimulation of 100 Hz was given for one second, to induce long-term potentiation, which was recorded as the accumulation potential for 60 minutes.

At the assessment step, the assay results of the accumulated potential at the candidate substance treatment step and the assay results of the accumulated potential at the control treatment step were analyzed and compared together for assessment. At the assessment step, the largest slope values of the individual accumulated potentials assayed over 20 minutes were averaged. When the average of the largest slope values in plurality was defined as 100% as basal slope value, the ratio of the largest slope value of accumulated potential assayed at each step to the basal slope value was assessed.

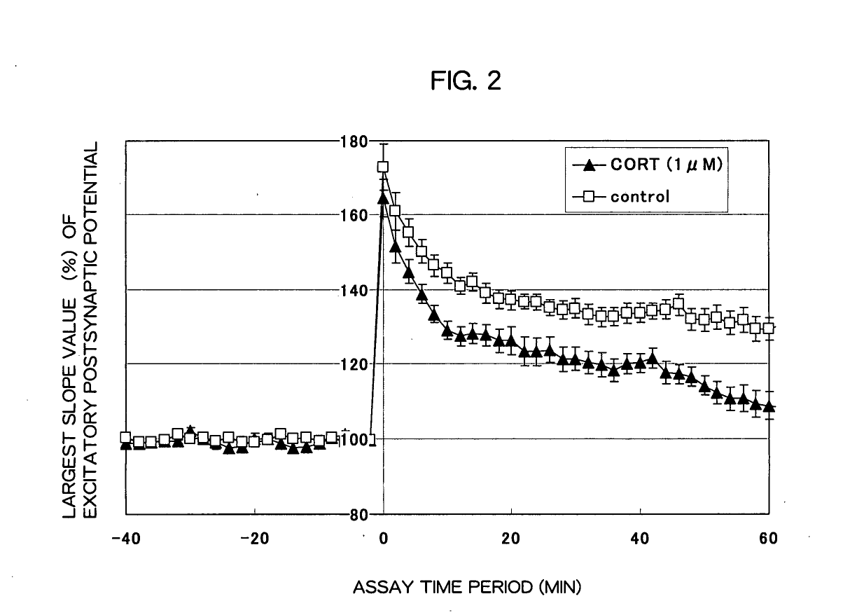

FIG. 2 shows the results of the assay of the long-term potentiation of excitatory postsynaptic potential (referred to as EPSP hereinafter) induced by the high-frequency stimulation in the non-treatment group and the control treatment group.

In FIG. 2, the horizontal axis shows assay time period (in minute) while the longitudinal axis shows the ratio of the largest slope value of accumulated potential assayed at each assay time period to the basal slope value defined as 100% as in above. Blank square mark (control) shows the assay results of the non-treatment group, while solid triangle mark (CORT) shows the assay results of the control treatment group. The long-term potentiation was induced by 100-Hz electric stimulation from the assay time zero (0) in FIG. 2 for one second.

As shown in FIG. 2, the potentiation of the EPSP reaction in hippocampus CA1 was observed from the assay time zero (0) in any of the hippocampus section of the non-treatment group and the hippocampus section of the control treatment group.

In the hippocampus section of the non-treatment group (blank square mark), the EPSP reaction was enhanced to about 130%, at the time of 60-minute high-frequency stimulation (at the assay time period of 60 minutes in FIG. 2). On the other hand, the EPSP reaction in the hippocampus section of the control treatment group (solid triangle mark) with the perfusion of the control solution containing corticosteron (1 μM) for 30 minutes before the high-frequency stimulation was reduced to about 110%, compared with the reaction in the hippocampus section of the non-treatment group, at the 60-minute time period after the high-frequency stimulation.

By putting nerve cells contained in the hippocampus sections used in the Example in contact to corticosteron, the level of the long-term potentiation [dimension (%) of EPSP reaction] was reduced by about 20%.

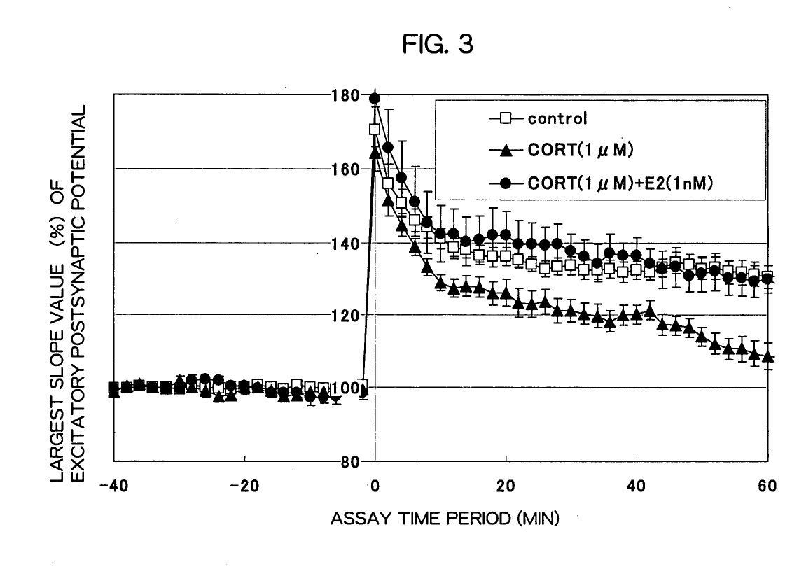

FIG. 3 shows the assay results of the long-term EPSP potentiation induced by the high-frequency stimulation in the candidate substance treatment group, in comparison with the assay results in FIG. 2. In FIG. 3, solid circle mark shows the assay results of the candidate substance treatment group.

As shown in FIG. 3, the EPSP reaction at the 60-minute time period after the high-frequency stimulation in the hippocampus section (solid circle mark) of the candidate substance treatment group with perfusion of the stimulation solution containing both corticosteron (1 μM) and estradiol (1 nM) over 30 minutes before the high-frequency stimulation (namely, the assay time period of −30 minutes to 0 minute) showed almost no difference from the EPSP reaction in the non-treatment group. The EPSP reaction was about 130%.

The reduction of the long-term potentiation level in case of the contact to corticosteron alone as verified in the hippocampus section of the control treatment group was nearly completely suppressed (in other words, dissolved) in the hippocampus section of the candidate substance treatment group in simultaneous contact to corticosteron and estradiol. The long-term potentiation level in the hippocampus section of the candidate substance treatment group was recovered at the same level as in the hippocampus section of the non-treatment group.

What is claimed is:

1. A screening method of a substance with an antidepressant action including
a cell preparation step for preparing a cell showing a predetermined electrophysiological response on a given stimulation and modifying the electrophysiological response when in contact to a depression-related substance,
a candidate substance treatment step for putting a candidate substance as a screening subject and the depression-related substance in contact to the prepared cell and assaying the electrophysiological response of the cell,
a control treatment step for putting the depression-related substance in contact to the prepared cell and assaying the modification of the electrophysiological response, and
an assessment step for assessing whether or not the modification of the electrophysiological response as assayed at the control treatment step is suppressed in the assay results obtained at the candidate substance treatment step.

2. A screening method of a substance with an antidepressant action according to claim 1, wherein the electrophysiological response is the membrane potential change of the cell on the basis of the given stimulation.

3. A screening method of a substance with an antidepressant action according to claim 1, wherein the cell is a nerve cell.

4. A screening method of a substance with an antidepressant action according to claim 3, wherein the nerve cell is a nerve cell contained in a brain tissue section of an animal.

5. A screening method of a substance with an antidepressant action according to claim 4, wherein the brain tissue section is a section of the hippocampus region.

6. A screening method of a substance with an antidepressant action according to claim 4, wherein the thickness of the brain tissue section is within a range of 150 μm to 400 μm.

7. A screening method of a substance with an antidepressant action according to claim 3, wherein the given stimulation is an electric stimulation and where the electrophysiological response is the change of an excitatory postsynaptic potential in association with the nerve cell on the basis of the electric stimulation.

8. A screening method of a substance with an antidepressant action according to claim 7, wherein the modification of the electrophysiological response due to the depression-related substance is the reduction of the long-term potentiation level to be assessed on the basis of the change of excitatory postsynaptic potential in association with the nerve cell on the basis of the electric stimulation and where it is assessed at the assessment step as to whether or not the reduction of the long-term potentiation level as assayed in the cell in contact to a depression-related substance at the control treatment step is suppressed in the cell in contact to the candidate substance and the depression-related substance at the candidate substance treatment step.

9. A screening method of a substance with an antidepressant action according to claim 1, wherein the depression-related substance is a stress hormone.

10. A screening method of a substance with an antidepressant action according to claim 9, wherein the stress hormone is corticoid or a derivative thereof.

11. A screening method of a substance with an antidepressant action according to claim 1, wherein putting the cell in contact to the candidate substance and the depression-related substance is carried out at the candidate substance treatment step by immersing the cell in a solution containing the candidate substance and the depression-related substance and where putting the cell in contact to the depression-related substance is carried out at the control treatment step by immersing the cell in a solution containing the depression-related substance.

12. A screening method of a substance with an antidepressant action according to claim 11, wherein the solution containing the candidate substance and the depression-related substance is perfused in a given container with the cell therein at the candidate substance treatment step and
where a solution containing the depression-related substance is perfused in a given container with the cell therein at the control treatment step.

13. A screening method of a substance with an antidepressant action according to claim 11, where the depression-related substance is corticosteron and where the concentration of the depression-related substance in the solution containing the candidate substance and the depression-related substance for use at the candidate substance treatment step and in the solution containing the depression-related substance for use at the control treatment step is 1 μM or more.
